(19) United States
(12) Patent Application Publication
(10) Pub. No.: US 2009/0317350 A1
(43) Pub. Date: Dec. 24, 2009

(54) KERATIN FIBRE COATING COMPOSITION COMPRISING AN AQUEOUS PHASE AND A TACKIFYING RESIN

(76) Inventors: Shao Xiang Lu, Plainsboro, NJ (US); Nathalie Jager-Lezer, Verrieres-Le-Buisson (FR); Balanda Atis, Newark, NJ (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 11/988,748

(22) PCT Filed: Jun. 22, 2006
(86) PCT No.: PCT/IB2006/002408

§ 371 Date: Apr. 17, 2009

Related U.S. Application Data
(60) Provisional application No. 60/699,023, filed on Jul. 13, 2005.

(51) Int. Cl. A61K 8/81; A61Q 1/10

(52) U.S. Cl. 424/70.7

(57) ABSTRACT

The present invention relates to a keratin fibre coating composition comprising an aqueous phase and at least one resin chosen from rosins, rosin derivatives and hydrocarbon-based resins, and mixtures thereof, the said resin having a number-average molecular weight of less than or equal to 10 000, the said composition being capable of forming a film with a water resistance such that ΔL is less than or equal to −1.

The present invention relates to the making up of keratin fibres, for instance the eyelashes, the eyebrows and the hair, and more particularly to making up the eyelashes.

The composition according to the invention may be in the form of a product for the eyelashes, or mascara, a product for the eyebrows, or a hair makeup product. The invention relates more especially to a mascara. It may especially be a makeup composition, a transparent or coloured composition to be applied over or under a makeup, also known, respectively, as a a “top coat” or a “base coat”, or alternatively an eyelash treatment composition.

In general, compositions for making up keratin fibres, and especially the eyelashes, of “emulsion mascara” type are in the form of an emulsion of waxes in an aqueous phase.

It so happens that the makeup film obtained with these compositions applied to the eyelashes has a tendency to become worn away over time: grains become deposited and leave marks around the eyes. Furthermore, the film thus weakened is not resistant to rubbing, especially with the fingers, and/or to water, for example during bathing or showering. The makeup is thus not resistant and shows poor staying power over time.

To improve the staying power of emulsion mascaras, it has been proposed, for example in document EP 1 016 418, to incorporate a film-forming polymer in the form of a dispersion of polymer particles in an aqueous phase (also known as a latex), but the incorporation of a latex in large amount leads to an increase in the consistency of the composition, which is reflected by difficulties in application, a reduced working time on the eyelashes and a granular, “board-like” deposit on the eyelashes.

The inventors have discovered, unexpectedly, that the incorporation of a particular resin into a composition with an aqueous phase allows the properties of the said composition to be improved, especially in terms of resistance to water, in particular to cold water (about 25° C.), and of resistance to rubbing.

The aim of the present invention is thus to propose another formulation route for a keratin fibre coating composition that has good properties in terms of resistance to water and/or to rubbing and that solves all or some of the problems associated with conventional formulation routes.

One subject of the present invention is, more specifically, a cosmetic composition for coating keratin fibres, comprising an aqueous phase and at least one resin chosen from rosin, rosin derivatives and hydrocarbon-based resins, and mixtures thereof, the said resin having a number-average molecular weight of less than or equal to 10 000.

The composition is advantageously capable of forming a film that has a water resistance such that ΔL is less than or equal to −1, for example ranging from −1 to −7, and ΔL is preferably less than or equal to −2, for example ranging from −2 to −6 and preferably from −3 to −5.

According to the present patent application, the term “water resistance” means the in vitro water resistance evaluated by colorimetry according to the following protocol:

The composition according to the invention is applied to 3 samples of straight 30-knots Caucasian hair (60 eyelashes 1 cm long, 2 cm fringe length) by performing three series of 10 sweeps at 2-minute intervals, with uptake of product between each series of 10. Each sample is then dried at room temperature for a drying time of one hour.

The three made-up samples are immersed in a container containing water, for 1 hour. The three samples are then wiped to and fro five times on a square cloth of the type such as Wypall L40 from Kimberley Clark.

To avoid variations in colour of the support, the measurement of L is performed as a “reference measurement”: the colour of the cloth is used as a white reference (L=96). The intensity of black deposited on the cloth by each sample is then measured using a calorimeter of the type such as CR 300 from Minolta.

Three measurements are taken on each mascara mark, and a mean value of L (mean L) is obtained.

The same makeup operation on the samples and luminosity measurement are performed with the following “control” composition:

| | |
|---|---|
| Carnauba wax | 7.3 |
| Rice bran wax | 7.45 |
| Candelilla wax | 2.5 |
| Esters of hydrogenated olive oil and of | 6.3 |
| stearyl alcohol (Phytowax Olive 18 L 57 | |
| from Sophim) | |
| Gum arabic | 1.52 |
| Hydroxyethylcellulose | 0.22 |
| Polyvinyl alcohol | 0.2 |
| Oxyethylenated (20 EO) oxypropylenated (20 PO) | 0.2 |
| polydimethylsiloxane (DC 2-5520) from | |
| Dow Corning) | |
| Simethicone | 0.12 |
| Black iron oxide | 8 |
| Stearic acid | 5.45 |
| Triethanolamine | 2.4 |
| Preserving agents | qs |
| Water | qs 100 |

The mean L for the control composition is obtained.

The (ΔL) corresponding to the difference between the mean L for the control composition and the mean L for the evaluated composition is then calculated.

The measurement taken on the colorimeter gives an indicative measurement of the “blackness” of the mascara mark: the blacker the mark, the closer the value (ΔL) is to 0. In other words, the further from 0 the value of (ΔL), the better the staying power, and vice versa.

A subject of the invention is also the use of at least one resin chosen from rosins, rosin derivatives and hydrocarbon-based resins, and mixtures thereof, the said resin having a number-average molecular weight of less than or equal to 10 000, in a keratin fibre coating composition comprising a continuous aqueous phase, to obtain a composition capable of forming a film which, when deposited on keratin fibres, has a water resistance of less than or equal to −1.

A subject of the present invention is also a process for making up keratin fibres, in which a composition as defined above is applied to the said keratin fibres and especially to the eyelashes.

The expression “at least one” means one or more individual compounds, and also mixtures thereof.

The composition according to the invention comprises a physiologically acceptable medium, especially a cosmetically acceptable medium, i.e. a medium that is compatible with keratin fibres such as the hair, the eyelashes and the eyebrows.

Resin

The resin used in the composition according to the invention has a number-average molecular weight of less than or equal to 10 000, especially ranging from 250 to 10 000, preferably less than or equal to 5000, especially ranging from 250 to 5000, better still less than or equal to 2000, especially ranging from 250 to 2000 and even better less than or equal to 1000, especially ranging from 250 to 1000.

The resin of the composition according to the invention is advantageously a tackifying resin. Such resins are described especially in the Handbook of Pressure Sensitive Adhesive, edited by Donatas Satas, 3rd edition, 1989, pp. 609-619.

The resin of the composition according to the invention is chosen from rosin, rosin derivatives and hydrocarbon-based resins, and mixtures thereof.

The rosin may be in the form of rosin gum or wood rosin, which are natural resins extracted from pine, or tall oil rosin (also known as tall oil glycerides).

These rosins are mixtures mainly comprising organic acids known as rosin acids (mainly acids of abietic type and of pimaric type).

The rosin derivatives may be derived in particular from the polymerization, hydrogenation and/or esterification (for example with polyhydric alcohols such as ethylene glycol, glycerol or pentaerythritol) of rosin acids. Examples that may be mentioned include the rosin esters sold under the reference Foral 85, Pentalyn H and Staybelite Ester 10 by the company Hercules; Sylvatac 95 and Zonester 85 by the company Arizona Chemical, or Unirez 3013 by the company Union Camp.

The hydrocarbon-based resins are chosen from low molecular weight polymers that may be classified, according to the type of monomer they comprise, as:
indene hydrocarbon-based resins such as the resins derived from the polymerization in major proportion of indene monomer and in minor proportion of monomers chosen from styrene, methylindene and methylstyrene, and mixtures thereof, these resins possibly being hydrogenated. These resins may have a molecular weight ranging from 290 to 1150.
Examples of indene resins that may be mentioned include those sold under the reference Escorez 7105 by the company Exxon Chem., Nevchem 100 and Nevex 100 by the company Neville Chem., Norsolene S105 by the company Sartomer, Picco 6100 by the company Hercules and Resinall by the company Resinall Corp., or the hydrogenated indene/methyl-styrene/styrene copolymers sold under the name “Regalite” by the company. Eastman Chemical, in particular Regalite R1100, Regalite R1090, Regalite R7100, Regalite R1010 Hydrocarbon Resin and Regalite R1125 Hydrocarbon Resin;
aliphatic pentanediene resins such as those derived from the majority polymerization of the 1,3-pentanediene (trans or cis-piperylene) monomer and of minor monomers chosen from isoprene, butene, 2-methyl-2-butene, pentene and 1,4-pentanediene, and mixtures thereof. These resins may have a molecular weight ranging from 1000 to 2500.
Such 1,3-pentanediene rosins are sold, for example, under the references Piccotac 95 by the company Eastman Chemical, Escorez 1304 by the company Exxon Chemicals, Nevtac 100 by the company Neville Chem. or Wingtack 95 by the company Goodyear;
mixed resins of pentanediene and of indene, which are derived from the polymerization of a mixture of pentanediene and indene monomers such as those described above, for instance the resins sold under the reference Escorez 2101 by the company Exxon Chemicals, Nevpene 9500 by the company Neville Chem., Hercotac 1148 by the company Hercules, Norsolene A 100 by the company Sartomer, and Wingtack 86, Wingtack Extra and Wingtack Plus by the company Goodyear;
diene resins of cyclopentadiene dimers such as those derived from the polymerization of first monomers chosen from indene and styrene, and of second monomers chosen from cyclopentadiene dimers such as dicyclopentadiene, methyldicyclopentadiene and other pentanediene dimers, and mixtures thereof. These resins generally have a molecular weight ranging from 500 to 800, for instance those sold under the reference Betaprene BR 100 by the company Arizona Chemical Co., Neville LX-685-125 and Neville LX-1000 by the company Neville Chem., Piccodiene 2215 by the company Hercules, Petro-Rez 200 by the company Lawter or Resinall 760 by the company Resinall Corp.;
diene resins of isoprene dimers such as terpenic resins derived from the polymerization of at least one monomer chosen from α-pinene, β-pinene and limonene, and mixtures thereof. These resins may have a molecular weight ranging from 300 to 2000. Such resins are sold, for example, under the names Piccolyte A115 and S125 by the company Hercules, and Zonarez 7100 or Zonatac 105 Lite by the company Arizona Chem.

Mention may also be made of certain modified resins such as hydrogenated resins, for instance those sold under the name Eastotac C6-C20 Polyolefin by the company Eastman Chemical Co., under the reference Escorez 5300 by the company Exxon Chemicals, or the resins Nevillac Hard or Nevroz sold by the company Neville. Chem., the resins Piccofyn A-100, piccotex 100 or Piccovar AP25 sold by the company Hercules or the resin SP-553 sold by the company Schenectady Chemical Co.

According to one preferred embodiment, the resin is chosen from indene hydrocarbon-based resins, in particular the hydrogenated indene/methylstyrene/styrene copolymers sold under the name “Regalite” by the company Eastman Chemical, such as Regalite R1100, Regalite R1090, Regalite R7100, Regalite R1010 Hydrocarbon Resin and Regalite R1125 Hydrocarbon Resin.

The resin may be present in the composition according: to the invention in a content ranging from 0.1% to 20% by weight, preferably from 0.5% to 15% by weight and better still from 1% to 10% by weight relative to the total weight of the composition.

Aqueous Phase

The aqueous phase of the composition according to the invention is advantageously a continuous aqueous phase.

The term “composition with a continuous aqueous phase” means that the composition has a conductivity, measured at 25° C., of greater than 23 μS/cm (microSiemens/cm), the conductivity being measured, for example, using an MPC227 conductimeter from Mettler Toledo and an Inlab730 conductivity measuring cell. The measuring cell is immersed in the composition so as to remove any air bubbles liable to form between the two electrodes of the cell. The conductivity reading is taken once the conductimeter value has stabilized. A mean is determined over at least three successive measurements.

The continuous aqueous phase of the composition according to the invention comprises water and/or at least cone water-soluble solvent.

In the present invention, the term “water-soluble solvent” denotes a compound that is liquid at room temperature and water-miscible (miscibility in water of greater than 50% by weight at 25° C. and atmospheric pressure).

The water-soluble solvents that may be used in the compositions according to the invention may also be volatile.

Among the water-soluble solvents that may be used in the compositions according to the invention, mention may be made especially of lower monoalcohols containing from 1 to 5 carbon atoms, such as ethanol and isopropanol, glycols containing from 2 to 8 carbon atoms, such as ethylene glycol, propylene glycol, 1,3-butylene glycol and dipropylene glycol, C₃ and C₄ ketones and C₂-C₄ aldehydes.

The aqueous phase (water and optionally the water-miscible solvent) may be present in a content ranging from 5% to 95% by weight, preferably ranging from 10% to 80% by weight and preferentially ranging from 15% to 60% by weight relative to the total weight of the composition.

Preferably, the aqueous phase represents at least 20% by weight, better still at least 30% and even better still at least 40% by weight relative to the total weight of the composition.

Emulsifying System

The composition according to the invention may contain emulsifying surfactants especially present in a proportion ranging from 0.1% to 30%, better still from 1% to 15% and better still from 2% to 10% by weight relative to the total weight of the composition.

According to the invention, an emulsifier appropriately chosen to obtain an oil-in-water emulsion is generally used. In particular, an emulsifier having at 25° C. an HLB (hydrophilic-lipophilic balance), in the Griffin sense, of greater than or equal to 8 may be used.

The HLB value according to Griffin is defined in J. Soc. Cosm. Chem. 1954 (volume 5), pages. 249-256.

These surfactants may be chosen from nonionic, anionic, cationic and amphoteric surfactants or combinations thereof. Reference may be made to the document “Encyclopedia of Chemical Technology, Kirk-Othmer”, volume 22, pp. 333-432, 3rd edition, 1979, Wiley, fort the definition of the properties and (emulsifying) functions of surfactants, in particular pp. 347-377 of this reference, for anionic, amphoteric and nonionic surfactants.

The surfactants preferably used in the composition according to the invention are chosen from:

a) nonionic surfactants with an HLB of greater than or equal to 8 at 25° C., used alone or as a mixture; mention may be made especially of:

oxyethylenated and/or oxypropylenated ethers (which may comprise from 1 to 150 oxyethylene and/or oxypropylene groups) of glycerol;

oxyethylenated and/or oxypropylenated ethers (which may comprise from 1 to −150 oxyethylene and/or oxypropylene groups) of fatty alcohols (especially of a C8-C24 and preferably C12-C18 alcohol), such as oxyethylenated cetearyl alcohol ether containing 30 oxyethylene groups (CTFA name Ceteareth-30) and the oxyethylenated ether of the mixture of C12-C15 fatty alcohols comprising 7 oxyethylene groups (CTFA name C12-15 Pareth-7 sold under the name Neodol 25-7® by Shell Chemicals);

fatty acid esters (especially of a C8-C24 and preferably C16-C22 acid) of polyethylene glycol (which may comprise from 1 to 150 ethylene glycol units), such as PEG-50 stearate and PEG-40 monostearate sold under the name Myrj 52P® by the company ICI Uniqema;

fatty acid esters (especially of a C8-C24 and preferably C16-C22 acid) of oxyethylenated and/or oxypropylenated glyceryl ethers (which may comprise from 1 to 150 oxyethylene and/or oxypropylene groups), for instance PEG-200 glyceryl monostearate sold under the name Simulsol 220® by the company SEPPIC; glyceryl stearate polyethoxylated with 30 ethylene oxide groups, for instance the product Tagat S® sold by the company Goldschmidt, glyceryl oleate polyethoxylated with 30 ethylene oxide groups, for instance the product Tagat O® sold by the company Goldschmidt, glyceryl cocoate polyethoxylated with 30 ethylene oxide groups, for instance the product Varionic LI 13® sold by the company Sherex, glyceryl isostearate polyethoxylated with 30 ethylene oxide groups, for instance the product Tagat O® sold by the company Goldschmidt, and glyceryl laurate polyethoxylated with 30 ethylene oxide groups, for instance the product Tagat I® from the company Goldschmidt;

fatty acid esters (especially of a C8-C24 and preferably C16-C22 acid) of oxyethylenated and/or oxypropylenated sorbitol ethers (which may comprise from 1 to 150 oxyethylene and/or oxypropylene groups), for instance polysorbate 60® sold under the name Tween 60® by the company Uniqema;

dimethicone copolyol, such as the product sold under the name Q2-5220® by the company Dow Corning;

dimethicone copolyol benzoate (Finsolv SLB 101® and 201® from the company Finetex);

copolymers of propylene oxide and of ethylene oxide, also known as EO/PO polycondensates;

and mixtures thereof.

The EO/PO polycondensates are more particularly copolymers consisting of polyethylene glycol and polypropylene glycol blocks, for instance polyethylene glycol/polypropylene glycol/polyethylene glycol triblock polycondensates. These triblock polycondensates have, for example, the following chemical structure:

H—(O—CH₂—CH₂)ₐ(O—CH(CH₃)—CH₂)_{b}—(O—CH₂—CH₂)ₐ—OH,

in which formula a ranges from 2 to 120 and b ranges from 1 to 100.

The EO/PO polycondensate preferably has a weight-average molecular weight ranging from 1000 to 15 000 and better still ranging from 2000 to 13 000. Advantageously, the said EO/PO polycondensate has a cloud point, at 10 g/l in distilled water, of greater than or equal to 20° C. and preferably greater than or equal to 60° C. The cloud point is measured according to ISO standard 1065. As EO/PO polycondensates that may be used according to the invention, mention may be made of the polyethylene glycol/polypropylene glycol/polyethylene glycol triblock polycondensates sold under the name Synperonic®, for instance Synperonic PE/L44® and Synperonic PE/F127®, by the company ICI.

b) nonionic surfactants with an HLB of less than 8 at 25° C., optionally combined with one or more nonionic surfactants with an HLB of greater than 8 at 25° C., such as those mentioned above, such as:

saccharide esters and ethers, such as sucrose stearate, sucrose cocoate and sorbitan stearate, and mixtures thereof, for instance Arlatone 2121® sold by the company ICI;

fatty acid esters (especially of a C8-C24 and preferably C16-C22 acid) of polyols, especially of glycerol or of sorbitol, such as glyceryl stearate, glyceryl stearate such as the product sold under the name. Tegin M® by the company Goldschmidt, glyceryl laurate such as the product sold under the name Imwitor 312® by the company Hüls, polyglyceryl-2 stearate, sorbitan tristearate or glyceryl ricinoleate;

the mixture of cyclomethicone/dimethicone copolyol sold under the name of Q2-3225® by the company Dow Corning.

c) anionic surfactants such as:

C₁₆-C₃₀ fatty acid salts, especially those derived from mines, for instance triethanolamine stearate;

polyoxyethylenated fatty acid salts, especially those derived from amines or alkali metal salts, and mixtures thereof;

phosphoric esters and salts thereof, such as DEA oleth-10 phosphate (Crodafos N 10N from the company Croda) or monocetyl monopotassium phosphate (Amphisol K from Givaudan);

sulfosuccinates such as Disodium PEG-5 citrate lauryl sulfosuccinate and Disodium ricinoleamido MEA sulfosuccinate;

alkyl ether sulfates, such as sodium lauryl ether sulfate;

isethionates;

acylglutamates such as Disodium hydrogenated tallow glutamate (Amisoft HS-21 R® sold by the company Ajinomato), and mixtures thereof.

Triethanolamine stearate is most particularly suitable for the invention. This surfactant is generally obtained by simple mixing of stearic acid and triethanolamine.

The compositions according to the invention may also contain one or more amphoteric surfactants, for instance N-acylamino acids such as N-alkylaminoacetates and disodium cocoamphodiacetate, and amine oxides such as stearamine oxide, or alternatively silicone surfactants, for instance dimethicone copolyol phosphates such as the product sold under the name Pecosil PS 100® by the company Phoenix Chemical.

### Hydrophilic Gelling Agent

The composition according to the invention may comprise a hydrophilic gelling agent.

The hydrophilic gelling agents that may be used in the compositions according to the invention may be chosen from:

homopolymers or copolymers of acrylic or methacrylic acid or the salts and esters thereof, and in particular the products sold under the names Versicol F® or Versicol K® by the company Allied Colloid, Ultrahold 8® by the company Ciba-Geigy, and the polyacrylic acids of Synthalen K type;

copolymers of acrylic acid and of acrylamide sold in the form of the sodium salt thereof under the name Reten® by the company Hercules, sodium polymethacrylate sold under the name Darvan 7® by the company Vanderbilt, and the sodium salts of polyhydroxycarboxylic acids sold under the name Hydagen F® by the company Henkel;

polyacrylic acid/alkyl acrylate copolymers of the Pemulen type;

AMPS (polyacrylamidomethylpropanesulfonic acid partially neutralized with ammonia and highly crosslinked) sold by the company Clariant;

AMPS/acrylamide copolymers of the Sepigel® or Simulgel® type, sold by the company SEPPIC, and

AMPS/polyoxyethylenated alkyl methacrylate copolymers (crosslinked or non-crosslinked), and mixtures thereof.

The water-soluble film-forming polymers mentioned above may also act as hydrophilic gelling agent.

The hydrophilic gelling agent may be present in the composition according to the invention in a solids content ranging from 0.01% to 60% by weight, preferably from 0.5% to 40% by weight, better still from 1% to 30% by weight or even from 5% to 20% by weight relative to the total weight of the composition.

### Liquid Fatty Phase

The composition according to the invention may comprise a fatty phase that is liquid at room temperature (25° C.) and atmospheric pressure (760 mmHg), composed of one or more mutually compatible non-aqueous fatty substances that are liquid at room temperature, also known as organic solvents or oils.

The oil may be chosen from volatile oils and/or non-volatile oils, and mixtures thereof.

For the purposes of the invention, the term “volatile oil” means an oil that is capable of evaporating on contact with the skin or the keratin fibre in less than one hour, at room temperature and atmospheric pressure. The volatile organic solvent(s) and volatile oils of the invention are volatile organic solvents and cosmetic oils that are liquid at room temperature, with a non-zero vapour pressure at room temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1300 Pa (0.01 to 10 mmHg). The term “non-volatile oil” means an oil that remains on the skin or the keratin fibre at room temperature and atmospheric pressure for at least several hours and that especially has a vapour pressure of less than 10⁻³ mmHg (0.13 Pa).

These oils may be hydrocarbon-based oils, silicone oils or fluoro oils, or mixtures thereof.

The term “hydrocarbon-based oil” means an oil mainly containing hydrogen and carbon atoms and optionally oxygen, nitrogen, sulfur or phosphorus atoms. The volatile hydrocarbon-based oils may be chosen from hydrocarbon-based oils containing from 8 to 16 carbon atoms, and especially branched C8-C16 alkanes, for instance C8-C16 isoalkanes of petroleum origin (also known as isoparaffins), for instance isododecane (also known as 2,2,4,4,6-pentamethylheptane), isodecane and isohexadecane, for example the oils sold under the trade names Isopar or Permethyl, branched C8-C16 esters and isohexyl neopentanoate, and mixtures thereof. Other volatile hydrocarbon-based oils, for instance petroleum distillates, especially those sold under the name Shell Solt by the company Shell, may also be used. The volatile solvent is preferably chosen from volatile hydrocarbon-based oils containing from 8 to 16 carbon atoms, and mixtures thereof.

volatile oils that may also be used include volatile silicones, for instance volatile linear or cyclic silicone oils, especially those with a viscosity ≦8 centistokes (8×10⁻⁶ m²/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Mention may also be made of the linear volatile alkyltrisiloxane oils of general formula (I):

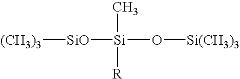

in which R represents an alkyl group containing from 2 to 4 carbon atoms and of which one or more hydrogen atoms may be substituted with one or more fluorine or chlorine atoms.

Among the oils of general formula (I) that may be mentioned are:
3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane,
   corresponding to the oils of formula (I) for which R is, respectively, a butyl group, a propyl group or an ethyl group.

Volatile fluorinated solvents such as nonafluoromethoxybutane or perfluoromethylcyclopentane may also be used.

The composition may also comprise at least one non-volatile oil, chosen in particular from non-volatile hydrocarbon-based oils and/or silicone oils and/or fluoro oils.

Non-volatile hydrocarbon-based oils that may especially be mentioned include:

hydrocarbon-based oils of plant origin, such as triesters of fatty acids and of glycerol, the fatty acids of which may have varied chain lengths from C4 to C24, these chains possibly being linear or branched, and saturated or unsaturated; these oils are especially wheatgerm oil, sunflower oil, grapeseed oil, sesame seed nil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, marrow oil, blackcurrant oil, evening primrose oil, millet oil, barley oil, quinoa oil, rye oil, safflower oil, candlenut oil, passionflower oil or musk rose oil; or caprylic/capric acid triglycerides, for instance those sold by the company Stéarineries Dubois or those sold under the names Miglyol 810, 812 and 818 by the company Dynamit Nobel;

synthetic ethers containing from 10 to 40 carbon atoms;

linear or branched hydrocarbons of mineral or synthetic origin, such as petroleum jelly, polydecenes, hydrogenated polyisobutene such as parleam, and squalane, and mixtures thereof;

synthetic esters, for instance oils of formula R₁COOR₂ in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂≧10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, alcohol or polyalcohol octanoates, decanoates or ricinoleates, for instance propylene glycol dioctanoate; hydroxylated esters, for instance isostearyl lactate or diisostearyl malate; and pentaerythritol esters;

fatty alcohols that are liquid at room temperature with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;

higher fatty acids such as oleic acid, linoleic acid or lincolenic acid;

carbonates;

acetates;

citrates;

and mixtures thereof.

The non-volatile silicone oils that may be used in the composition according to the invention may be non-volatile polydimethylsiloxanes (PDMS), polydimethylsiloxaries comprising alkyl or alkoxy groups, which are pendent and/or at the end of a silicone chain, these groups each containing from 2 to 24 carbon atoms, phenyl silicones, for instance phenyl trimethicones, phenyl dimethicones, phenyltrimethylsiloxydiphenylsiloxanes, diphenyldimethicones, diphenylmethyldiphenyltrisiloxanes and 2-phenylethyltrimethylsiloxysilicates.

The fluoro oils that may be used in the invention are especially fluorosilicone oils, fluoro polyethers and fluorosilicones as described in document EP-A-847 752.

According to one embodiment, the fatty phase advantageously contains an ester oil. This ester oil may be chosen from the esters of monocarboxylic acids with monoalcohols and polyalcohols.

Advantageously, the said ester corresponds to formula (I) below:

R₁—CO—O—R₂ (I)

where R₁ represents a linear or branched alkyl radical of 1 to 40 carbon atoms and preferably of 7 to 19 carbon atoms, optionally comprising one or more ethylenic double bonds, and optionally substituted,

R₂ represents a linear or branched alkyl radical of 1 to 40 carbon atoms, preferably of 3 to 30 carbon atoms and better still of 3 to 20 carbon atoms, optionally comprising one or more ethylenic double bonds, and optionally substituted.

The term “optionally substituted” means that R₁ and/or R₂ can bear one or more substituents chosen, for example, from groups comprising one or more hetero atoms chosen from O, N and S, such as amino, amine, alkoxy and hydroxyl.

Preferably, the total number of carbon atoms of R₁+R₂ is ≧9.

R₁ may represent the residue of a linear or, preferably, branched fatty acid, preferably a higher fatty acid, containing from 1 to 40 and even better from 7 to 19 carbon atoms, and R₂ may represent a linear or, preferably, branched hydrocarbon-based chain containing from 1 to 40, preferably from 3 to 30 and even better from 3 to 20 carbon atoms. Once again, preferably the number of carbon atoms of R₁+R₂≧9.

Examples of groups R₁ are those derived from fatty acids chosen from the group consisting of acetic acid, propionic acid, butyric acid, caproic acid, caprylic acid, pelargonic acid, capric acid, undecanoic acid, lauric acid, myristic acid, palmitic acid, stearic acid, isostearic acid, arachidic acid, behenic acid, oleic acid, linolenic acid, linoleic acid, oleostearic acid, arachidonic acid and erucic acid, and mixtures thereof.

Examples of esters, include purcellin oil (cetostearyl octanoate), isononyl isononanoate, isopropyl myristate, 2-ethylhexyl palmitate, 2-octyldodecyl stearate, 2-octyldodecyl erucate, isostearyl isostearate, and heptanoates, octanoates, decanoates or ricinoleates of alcohols or polyalcohols, for example of fatty alcohols.

Advantageously, the esters are chosen from the compounds of formula (I) above, in which R₁ represents an unsubstituted linear or branched alkyl group of 1 to 40 carbon atoms and preferably of 7 to 19 carbon atoms, optionally comprising one or more ethylenic double bonds, and R₂ represents an unsubstituted linear or branched alkyl group of 1 to 40 carbon atoms, preferably of 3 to 30 carbon atoms and even better of 3 to 20 carbon atoms, optionally comprising one or more ethylenic double bonds.

Preferably, R₁ is an unsubstituted branched alkyl group of 4 to 14 carbon atoms and preferably of 8 to 10 carbon atoms, and R₂ is an unsubstituted branched alkyl group of 5 to 15 carbon atoms and preferably of 9 to 11 carbon atoms. Preferably, in formula (I), R₁—CO— and R₂ have the same number of carbon atoms and are derived from the same radical, preferably an unsubstituted branched alkyl, for example isononyl, i.e. the ester oil molecule is advantageously symmetrical.

The ester oil will preferably be chosen from the following compounds:

isononyl isononanoate,

cetostearyl octanoate,

isopropyl myristate,

2-ethylhexyl palmitate,

2-octyldodecyl stearate,

2-octyldodecyl erucate,

isostearyl isostearate.

The liquid fatty phase may represent from 0.5% to 30% by weight, preferably from 1% to 20% and even more preferably from 2% to 10% by weight relative to the total weight of the composition.

### Structuring Agent

The composition according to the invention may comprise at least one agent for structuring the oily phase or organic solvent (formed from the volatile or non-volatile organic, solvents or oils described above), chosen from waxes, semi-crystalline polymers and lipophilic gelling agents and thickeners, and mixtures thereof.

The structuring agent may represent from 0.05% to 70% by weight, preferably from 0.1% to 40%, better still from 1% to 30% by weight and even more preferably from 1.5% to 15% by weight relative to the total weight of the composition.

The amount of oily structuring agent may be adjusted by a person skilled in the art as a function of the structuring properties of the said agents.

### Waxes

The wax under consideration in the context of the present invention is generally a lipophilic compound that is solid at room temperature (25° C.) which may or may not be deformable, with a solid/liquid reversible change of state, having a melting point of greater than or equal to 30° C., which may be up to 200° C. and in particular up to 120° C.

By bringing the wax to the liquid form (melting), it is possible to make it miscible with oils and to form a microscopically uniform mixture, but on cooling the mixture to room temperature, recrystallization of the wax in the oils of the mixture is obtained.

In particular, the waxes that are suitable for the invention may have a melting point of greater than or equal to 45° C. and in particular greater than or equal to 55° C.

For the purposes of the invention, the melting point corresponds to the temperature of the most endothermic peak observed by thermal analysis (DSC) as described in ISO standard 11357-3; 1999. The melting point of the wax may be measured using a differential scanning calorimeter (DSC), for example the calorimeter sold under the name MDSC 2920 by the company TA Instruments.

The measuring protocol is as follows:

A sample of 5 mg of wax placed in a crucible is subjected to a first temperature rise ranging from −20° C. to 100° C., at a heating rate of 10° C./minute, it is then cooled from 100° C. to −20° C. at a cooling rate of 10° C./minute and is finally subjected to a second temperature increase ranging from −20° C. to 10° C. at a heating rate of 5° C./minute. During the second temperature increase, the variation of the difference in power absorbed by the empty crucible and by the crucible containing the sample of wax is measured as a function of the temperature. The melting point of the compound is the temperature value corresponding to the top of the peak of the curve representing the variation in the difference in absorbed power as a function of the temperature.

The waxes that may be used in the compositions according to the invention are chosen from waxes that are solid at room temperature of animal, plant, mineral or synthetic origin, and mixtures thereof.

The waxes that may be used in the compositions according to the invention generally have a hardness ranging from 0.01 MPa to 15 MPa, especially greater than 0.05 MPa and in particular greater than 0.1 MPa.

The hardness is determined by measuring the compression force, measured at 20° C. using the texturometer sold under the name TA-XT2 by the company Rheo, equipped with a stainless-steel cylindrical spindle 2 mm in diameter, travelling at a measuring speed of 0.1 mm/second, and penetrating the wax to a penetration depth of 0.3 mm.

The measuring protocol is as follows:

The wax is melted at a temperature equal to the melting point of the wax+10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C. before measuring the hardness or the tack.

The texturometer spindle is displaced at a speed of 0.1 mm/s then penetrates the wax to a penetration depth of 0.3 mm. When the spindle has penetrated the wax to a depth of 0.3 mm, the spindle is held still for 1 second (corresponding to the relaxation time) and is then withdrawn at a speed of 0.5 mm/s.

The hardness value is the maximum compression force measured divided by the area of the texturometer cylinder in contact with the wax.

As illustrations of waxes that are suitable for the invention, mention may be made especially of hydrocarbon-based waxes, for instance beeswax, lanolin wax and Chinese insect waxes; rice bran wax, carnauba wax, candelilla wax, ouricury wax, alfalfa wax, berry wax, shellac wax, Japan wax and sumach wax; montan wax, orange wax, lemon wax, microcrystalline waxes, paraffins and ozokerite; polyethylene waxes, the waxes obtained by Fischer-Tropsch synthesis and waxy copolymers, and also esters thereof.

Mention may also be made of waxes obtained by catalytic hydrogenation of animal or plant oils containing linear or branched C₈-C₃₂ fatty chains. Among these waxes that may especially be mentioned are isomerized jojoba oil such as the trans-isomerized partially hydrogenated jojoba oil manufactured or sold by the company Desert Whale under the commercial reference Iso-Jojoba-50®, hydrogenated sunflower oil, hydrogenated castor oil, hydrogenated coconut oil, hydrogenated lanolin oil and bis(1,1,1-trimethylolpropane) tetrastearate sold under the narne Hest 2T-4S® by the company Heterene.

Mention may also be made of silicone waxes and fluoro waxes.

The waxes obtained by hydrogenation, of castor oil esterified with cetyl alcohol, sold under the names Phytowax ricin 16L64® and 22L73® by the company Sophim, may also be used. Such waxes are described in patent application FR-A-2 792 190.

According to one particular embodiment, the compositions according to the invention may comprise at least one “tacky” wax, i.e. a wax with a tack of greater than or equal to 1.7 N.s and a hardness of less than or equal to 3.5 MPa.

The tacky wax used may especially have a tack ranging from 0.1 N.s to 10 N.s, in particular ranging, from 0.1 N.s to 5 N.s, preferably ranging from 0.2 N.s to 5 N.s and better still ranging from 0.3 N.s to 2 N.s.

The tack of the wax is determined by measuring the change in the force (compression force) as a function of time, at 20° C. according to the protocol indicated above for the hardness.

During the 1-second relaxation time, the force (compression force) decreases greatly until it becomes zero, and then, during the withdrawal of the spindle, the force (stretching force) becomes negative and then rises again to the value 0. The tack corresponds to the integral of the curve of the force as a function of time for the part of the curve corresponding to negative values of the force. The tack value is expressed in N.s.

The tacky wax that may be used generally has a hardness of less than or equal to 3.5 MPa, in particular ranging from 0.01 MPa to 3.5 MPa, especially ranging from 0.05 MPa to 3 MPa.

Tacky waxes that may be used include a C₂₀-C₄₀ alkyl (hydroxystearyloxy)stearate (the alkyl group containing from 20 to 40 carbon atoms), alone or as a mixture.

Such a wax is especially sold under the names Kester Wax K 82 P®, and, Kester Wax K 80 P® by the company Koster Keunen.

In the present invention, waxes provided in the form of small particles having a diameter expressed as the mean “effective” volume diameter D[4.3] of about from 0.5 to 30 micrometres, in particular from 1 to 20 micrometres and more particularly from 5 to 10 micrometres, which are referred to hereinafter as “microwaxes”, may also be used.

The particle sizes may be measured by various techniques; mention may be made in particular of light-scattering techniques (dynamic and static), Coulter counter methods, sedimentation rate measurements (related to the size via Stokes' law) and microscopy. These techniques make it possible to measure a particle diameter and, for some of them, a particle size distribution.

The sizes and size distributions of the particles in the compositions according to the invention are preferably measured by static light scattering using a commercial granulometer such as the MasterSizer 2000 from Malvern. The data are processed on the basis of the Mie scattering theory. This theory, which is exact for isotropic particles, makes it possible to determine an “effective” particle diameter in the case of non-spherical particles. This theory is described especially in the publication by Van de Hulst, H. C., “Light Scattering by Small Particles,” Chapters 9 and 10, Wiley, New York, 1957.

The composition is characterized by its mean “effective” diameter by volume D[4.3], defined in the following manner:

$$D [ 4.3 ] = \frac{{\sum }_{i} {V}_{i} \cdot {d}_{i}}{{\sum }_{i} {V}_{i}}$$

in which Vᵢ represents the volume of the particles with an effective diameter dᵢ. This parameter is described especially in the technical documentation of the grapulometer.

The measurements are performed at 25° C. on a dilute particle dispersion, obtained from the composition in the following manner: 1) dilution by a factor of 100 with water, 2) homogenization of the solution, 3) standing of the solution for 18 hours, 4) recovery of the whitish uniform supernatant.

The “effective” diameter is obtained by taking a refractive index of 1.33 for water and a mean refractive index of 1.42 for the particles.

As microwaxes that may be used in the compositions according to the invention, mention may be made of carnauba microwaxes, such as the product sold under the name MicroCare 350® by the company Micro Powders, synthetic microwaxes, such as the product sold under the name MicroEase 114S® by the company Micro Powders, microwaxes consisting of a mixture of carnauba wax and polyethylene wax, such as the products sold under the names Micro Care 300® and 310® by the company Micro Powders, microwaxes consisting of a mixture of carnauba wax and of synthetic wax, such as the product sold under the name Micro Care 325® by the company Micro Powders, polyethylene microwaxes, such as the products sold under the names Micropoly 200®, 220®, 220L® and 250S® by the company Micro Powders, and polytetrafluoroethylene microwaxes such as the products sold under the names Microslip 519® and 519 L® by the company Micro Powders.

### Semi-Crystalline Polymers

The term “polymer” means compounds containing, at least two repeating units, preferably at least three repeating units and more especially at least ten repeating units. The term “semi-crystalline polymer” means polymers comprising a crystallizable portion, a crystallizable side chain or a crystallizable block in the skeleton, and an amorphous portion in the skeleton and having a first-order reversible phase-change temperature, in particular of melting (solid-liquid transition). When the crystallizable portion is in the form of a crystallizable block of the polymer skeleton, the amorphous portion of the polymer is in the form of an amorphous block; in this case, the semi-crystalline polymer is a block copolymer, for example, of the diblock, triblock or multiblock type, comprising at least one crystallizable block and at least one amorphous block. The term “block” generally means at least five identical repeating units. The crystallizable block(s) is (are) of chemical nature different than that of the amorphous block(s).

The semi-crystalline polymer has a melting point of greater than or equal to 30° C. (especially ranging from 30° C. to 80° C.), preferably ranging from 30° C. to 60° C. This melting point is a first-order change of state temperature.

This melting point may be measured by any known method and in particular using a differential scanning calorimeter (DSC).

Advantageously, the semi-crystalline polymer(s) to which the invention applies have a number-average molecular mass of greater than or equal to 1000. Advantageously, the semi-crystalline polymer(s) of the composition of the invention have a number-average molecular mass M̅n ranging from 2000 to 800 000, preferably from 3000 to 500 000, better still from 4000 to 150 000, especially less than 100 000 and better still from 4000 to 99 000. Preferably, they have a number-average molecular mass of greater than 5600, for example ranging from 5700 to 99 000. For the purposes of the invention, the term “crystallizable chain or block” means a chain or block which, if it were alone, would reversibly change from the amorphous state to the crystalline state, depending on whether the system is above or below the melting point. For the purposes of the invention, a chain is a group of atoms, which is pendent or lateral relative to the polymer skeleton. A block is a group of atoms belonging to the skeleton, this group constituting one of the repeating units of the polymer. Advantageously, the “crystallizable side chain” may be a chain containing at least six carbon atoms.

The semi-crystalline polymer may be chosen from block copolymers comprising at least one crystallizable block and at least one amorphous block, and homopolymers and copolymers bearing at least one crystallizable side chain per repeating unit, and mixtures thereof.

Such polymers are described, for example, in document EP 1 396 259.

A. Semi-Crystalline Polymers Containing Crystallizable Side Chains

Mention may be made in particular of those defined in documents U.S. Pat. No. 5,156,911 and WO-A-01/19333. They are homopolymers or copolymers comprising from 50% to 100% by weight of units resulting from the polymerization of one or more monomers bearing a crystallizable hydrophobic side chain.

These homopolymers or copolymers are of any nature, provided that they meet the conditions mentioned previously.

B. Polymers Bearing in the Skeleton at Least One Crystallizable Block

These polymers are especially block copolymers consisting of at least two blocks of different chemical nature, one of which is crystallizable.

The block polymers defined in U.S. Pat. No. 5,156,911 may be used;

The block copolymers of olefin or of cycloolefin containing a crystallizable chain, for instance those derived from the block polymerization of:

cyclobutene, cyclohexene, cyclooctene, norbornene (i.e. bicyclo(2,2,1)-2-heptene), 5-methylnorbornene, 5-ethylnorbornene, 5,6-dimethylnorbornene, 5,5,6-trimethyorbornene, 5-ethylidenenorbornene, 5-phenylnorbornene, 5-benzylnorbornene, 5-vinylnorbornene, 1,4,5,8-dimethano-1,2,3,4,4a,5,8a-octahydronaphthalene, dicyclopentadiene, or mixtures thereof,

with ethylene, propylene, 1-butene, 3-methyl-1-butene, 1-hexene, 4-methyl-1-pentene, 1-octene, 1-decene or 1-eicosene, or mixtures thereof,

and in particular copoly(ethylene/norbornene) blocks and (ethylene/propylene/ethylidene-norbornene) block terpolymers. Those resulting from the block copolymerization of at least two C₂-C₁₆, better still C₂-C₁₂ and even better Still C₄-C₁₂ α-olefins such as those mentioned above and in particular block bipolymers of ethylene and of 1-octene may also be used.

The copolymers may be copolymers containing at least one crystallizable block, the rest of the copolymer being amorphous (at room temperature). These copolymers may also contain two crystallizable blocks of different chemical nature. The preferred copolymers are those that simultaneously contain at room temperature a crystallizable block and an amorphous block that are both hydrophobic and lipophilic, sequentially distributed; mention may be made, for example, of polymers containing one of the crystallizable blocks and one of the amorphous blocks below:

Block that is crystallizable by nature: a) of polyester type, for instance poly(alkylene terephthalate), b) of polyolefin type, for instance polyethylenes or polypropylenes.

Amorphous and lipophilic block, for instance amorphous polyolefins or copoly(olefin)s such as poly(isobutylene), hydrogenated, polybutadiene or hydrogenated poly(isoprene).

As examples of such copolymers containing a crystallizable block and an amorphous block, mention may be made of:

α) poly(ε-caprolactone)-b-poly(butadiene) block copolymers, preferably used hydrogenated, such as those described in the article “Melting behavior of poly (ε-caprolactone)-block-polybutadiene copolymers” from S. Nojima, Macromolecules, 32, 3727-3734 (1999),

β) the hydrogenated block or multiblock poly(butylene terephthalate)-b-poly(isoprene) block copolymers cited in the article “Study of morphological and mechanical properties of PP/PBT” by B. Boutevin et al., Polymer Bulletin, 34, 117-123 (1995),

γ) the poly(ethylene)-b-copoly(ethylene/propylene) block copolymers cited in the articles “Morphology of semi-crystalline block copolymers of ethylene-(ethylene-alt-propylene)” by P. Rangarajan et al., Macromolecules, 26, 4640-4645 (1993) and “Polymer aggregates with crystalline cores: the system poly(ethylene)-poly(ethylene-propylene)” by P. Richter et al., Macromolecules, 30, 1053-1068 (1997),

δ) the poly (ethylene)-b-poly(ethylethylene) block copolymers cited in the general article “Crystallization in block copolymers” by I. W. Hamley, Advances in Polymer Science, Vol. 148, 113-137 (1999).

Preferably, the semi-crystalline polymers in the composition according to the invention are non-crosslinked.

According to one particular embodiment of the invention, the polymer is chosen from copolymers resulting from the polymerization of at least one monomer containing a crystallizable chain chosen from saturated C₁₄ to C₂₄ alkyl (meth)acrylates, C₁₁ to C₁₅ perfluoroalkyl (meth)acrylates, C₁₄ to C₂₄ N-alkyl(meth)acrylamides with or without a fluorine atom, vinyl esters containing C₁₄ to C₂₄ alkyl or perfluoroalkyl chains, vinyl ethers containing C₁₄ to C₂₄ alkyl or perfluoralkyl chains, C₁₄ to C₂₄ α-olefins, para-alkylstyrenes with an alkyl group containing from 12 to 24 carbon atoms, with at least one optionally; fluorinated C₁ to C₁₀ monocarboxylic acid ester or amide, which may be represented by the following formula:

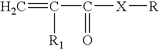

in which R₁ is H or CH₃, R represents an optionally fluorinated C₁-C₁₀ alkyl group and X represents O, NH or NR₂, in which R₂ represents an optionally fluorinated C₁-C₁₀ alkyl group.

According to a more particular embodiment of the invention, the polymer is derived from a monomer containing a crystallizable chain chosen from saturated C₁₄-C₂₂ alkyl (meth)acrylates.

As a particular example of a semi-crystalline polymer that may be used in the composition according to the invention, mention may be made of the Intelimer® products from the company Landec described in the brochure “Intelimer® Polymers”, Landec IP22 (Rev. 4-97). These polymers are in solid form at room temperature (25° C.). They bear crystallizable side chains and have the above formula X.

### Lipophilic Gelling Agents or Thickeners

The gelling agents that may be used in the compositions according to the invention may be organic or mineral, polymeric or molecular lipophilic gelling agents.

Mineral lipophilic gelling agents that may be mentioned include optionally modified clays, for instance hectorites modified with a C₁₀ to C₂₂ fatty acid ammonium chloride, for instance hectorite modified with disteayldimethylammonium chloride, for instance the product sold under the name Bentone 38V® by the company Elementis.

Mention may also be made of fumed silica optionally subjected to a hydrophobic surface treatment, the particle size of which is less than 1 μm. Specifically, it is possible to chemically modify the surface of the silica, by chemical reaction generating a reduced number of silanol groups present at the surface of the silica. It is especially possible to substitute silanol groups with hydrophobic groups: a hydrophobic silica is then obtained. The hydrophobic groups may be:

trimethylsiloxyl groups, which are obtained especially by treating fumed silica in the presence of hexamethyldisilazane. Silicas thus treated are known as “silica silylate” according to the CTFA (6th edition, 1995). They are sold, for example, under the references Aerosil R812® by the company Degussa, and Cab-O-Sil TS-530® by the company Cabot;

dimethylsilyloxyl or polydimethylsiloxane groups, which are obtained especially by treating fumed silica in the presence of polydimethylsiloxane or dimethyldichlorosilane. Silicas thus treated are known as “silica dimethyl silylate” according to the CTEA (6th edition, 1995). They are sold, for example, under the references Aerosil R972® and Aerosil R974® by the company Degussa, and Cab-O-Sil TS-610® and Cab-O-Sil TS-720® by the company Cabot.

The hydrophobic fumed silica particularly has a particle size that may be nanometric to micrometric, for example ranging from about 5 to 200 nm.

It is also possible to use non-polymeric, molecular organic gelling agents, also known as organogelling agents, associated with a liquid fatty phase (which may be the liquid fatty phase of the composition according to the invention), which are compounds whose molecules are capable of establishing between themselves physical interactions leading to self-aggregation of the molecules with formation of a supramolecular 3D network that is responsible for the gelation of the liquid fatty phase.

The supramolecular network may result from the formation of a network of fibrils (caused by the stacking or aggregation of organogelling molecules), which immobilizes the molecules of the liquid fatty phase.

The ability to form this network of fibrils, and thus to gel, depends on the nature (or chemical class) of the organogelling agent, on the nature of the substituents borne by its molecules for a given chemical class, and on the nature of the liquid fatty phase.

The physical interactions are of diverse nature but exclude co-crystallization. These physical interactions are in particular interactions of self-complementary hydrogen interaction type, π interactions between unsaturated rings, dipolar interactions, coordination bonds with organometallic derivatives, and combinations thereof. In general, each molecule of an organogelling agent can establish several types of physical interaction with a neighbouring molecule. Thus, advantageously, the molecules of the organogelling agents according to the invention comprise at least one group capable of establishing hydrogen bonds and better still at least two groups, at least one aromatic ring and better still at least two aromatic rings, at least one or more ethylenically unsaturated bonds and/or at least one or more asymmetric carbons. Preferably, the groups capable of forming hydrogen bonds are chosen from hydroxyl, carbonyl, amine, carboxylic acid, amide, urea and benzyl groups, and combinations thereof.

The organogelling agent(s) according to the invention is (are) soluble in the liquid fatty phase after heating to obtain a transparent uniform liquid phase. They may be solid or liquid at room temperature and atmospheric pressure.

The molecular organogelling agent(s) that may be used in the composition according to the invention is (are) especially those described in the document “Specialist Surfactants” edited by D. Robb, 1997, pp. 209-263, Chapter 8 by P. Terech, European patent applications EP-A-1 068 854 and EP-A-1 086 945, or alternatively in patent application WO-A-02/47031.

Mention may be made especially, among these organogelling agents, of amides of carboxylic acids, in particular of tricarboxylic acids, for instance cyclohexanetricarboxamides (see European patent application EP-A-1 068 854), diamides with hydrocarbon-based chains each containing from 1 to 22 carbon atoms, for example from 6 to 18 carbon atoms, the said chains being unsubstituted or substituted with at least one substituent chosen from ester, urea and fluoro groups (see patent application EP-A-1 086 945) and especially diamides resulting from the reaction of diaminocyclohexane, in particular diaminocyclohexane in trans form, and of an acid chloride, for instance N,N′-bis-(dodecanoyl)-1,2-diaminocyclohexane, N-acylamino acid amides, for instance the diamides resulting from the action of an N-acylamino acid with amines containing from 1 to 22 carbon atoms, for instance those described in document WO-93/23008 and especially N-acylglutamic acid amides in which the acyl group represents a C₈ to C₂₂ alkyl chain, such as N-lauroyl-L-glutamic acid dibutylamide, manufactured or sold by the company Ajinomoto under the name GP-1, and mixtures thereof.

The polymeric organic lipophilic gelling agents or thickeners are, for example:
partially or totally crosslinked elastomeric organopolysiloxanes of three-dimensional structure, for instance those sold under the names KSG6®, KSG16® and KSG18® from Shin-Etsu, Trefil E-505C® or Trefil E-506C® from Dow Corning, Gransil SR-CYC®, SR DMF 10®, SR-DC556®, SR 5CYC gel®, SR DMF 10 gel® and SR D 556 gel® from Grant Industries and SF 1204® and JK 113® from General Electric;
ethylcellulose, for instance the product sold under the name Ethocel® by Dow Chemical;
polycondensates of polyamide type resulting from condensation between (α) at least one acid chosen from dicarboxylic acids containing at least 32 carbon atoms, such as fatty acid dimers, and (β) an alkylenediamine and in particular ethylenediamine, in which the polyamide polymer comprises at least one carboxylic acid end group esterified or amidated with at least one saturated and linear monoalcohol or one saturated and linear monoamine containing from 12 to 30 carbon atoms, and in particular ethylenediamine/stearyl dilinoleate copolymers such as the product sold under the name Uniclear 100 VG® by the company Arizona Chemical;
silicone polymers of the type such as:

1) polyorganosiloxanes comprising at least two groups capable of establishing hydrogen interactions, these two groups being located in the polymer chain, and/or

2) polyorganosiloxanes comprising at least two groups capable of establishing hydrogen interactions, these two groups being located on grafts or branches.

The groups capable of establishing hydrogen interactions may be chosen from ester, amide, sulfonamide, carbamate, thiocarbamate, urea, urethane, thiourea, oxamido, guanidino and biguanidino groups, and combinations thereof.

The silicone polymers used as structuring agents in the composition of the invention are polymers of the polyorganosiloxane type, for instance those described in documents U.S. Pat. No. 5,874,069, U.S. Pat. No. 5,919,441, U.S. Pat. No. 6,051,216 and U.S. Pat. No. 5,981,680.

In particular, the silicone polymers are polyorganosiloxanes as defined above in which the units capable of establishing hydrogen interactions are located in the polymer chain.

The silicone polymers may be more particularly polymers comprising at least one unit corresponding to the general formula I:

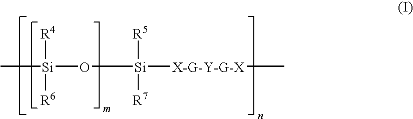

in which:

1) R⁴, R⁵, R⁶ and R⁷, which may be identical or different, represent a group chosen from:

linear, branched or cyclic, saturated or unsaturated, C₁ to C₄₀ hydrocarbon-based groups, possibly containing in their chain one or more oxygen, sulfur and/or nitrogen atoms, and possibly being partially or totally substituted with fluorine atoms,

C₆ to C₁₀ aryl groups, optionally substituted with one or more C₁ to C₄ alkyl groups,

polyorganosiloxane chains possibly containing one or more oxygen, sulfur and/or nitrogen atoms;

2) the groups X, which may be identical or different, represent a linear or branched C₁ to C₃₀ alkylenediyl group, possibly containing in its chain, one or more oxygen and/or nitrogen atoms;

3) Y is a saturated or unsaturated, C₁ to C₅₀ linear or branched divalent alkylene, arylene, cycloalkylene, alkylarylene or arylalkylene group, possibly comprising one or more oxygen, sulfur and/or nitrogen atoms, and/or bearing as substituent one of the following atoms or groups of atoms: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl, C₁ to C₃ hydroxyalkyl and C₁ to C₆ aminoalkyl groups; or

4) Y represents a group corresponding to the formula:

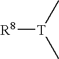

in which

T represents a linear or branched, saturated or unsaturated, C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and

R⁸ represents a linear or branched C₁ to C₅₀ alkyl group or a polyorganosiloxane chain, possibly comprising one or more ester, amide, urethane, thiocarbamate, urea, thiourea and/or sulfonamide groups, which may possibly be linked to another chain of the polymer;

5) the groups G, which may be identical or different, represent divalent groups chosen from:

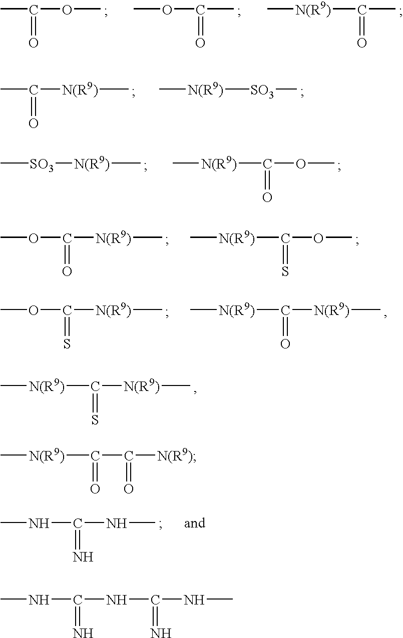

in which R⁹ represents a hydrogen atom or a linear or branched C₁ to C₂₀ alkyl group, on condition that at least 50% of the groups R⁹ of the polymer represent a hydrogen atom and that at least two of the groups G of the polymer are a group other than:

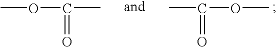

6) n is an integer ranging from 2 to 500 and preferably from 2 to 200, and m is an integer ranging from 1 to 1000, preferably from, 1 to 700 and better still from 6 to 200.

According to the invention, 80% of the groups R⁴, R⁵, R⁶ and R⁷ of the polymer are preferably chosen from methyl, ethyl, phenyl and 3,3,3-trifluoropropyl groups.

According to a first advantageous embodiment, the groups capable of establishing hydrogen interactions are amide groups of formulae —C(O)NH— and —HN—C(O)—.

In this case, the structuring agent may be a polymer comprising at least one unit of formula (III) or (IV):

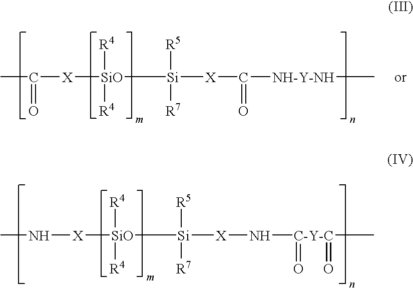

in which R⁴, R⁵, R⁶, R⁷, X, Y, m and n are as defined above.

In these polyamides of formula (III) or (IV), m is in the range from 1 to 700, in particular from 15 to 500 and especially from 50 to 200, and n is in particular in the range from 1 to 500, preferably from 1 to 100 and better still from 4 to 25,

X is preferably a linear or branched alkylene chain containing from 1 to 30 carbon atoms, in particular 1 to 20 carbon atoms, especially from 5 to 15 carbon atoms and more particularly 10 carbon atoms, and

Y is preferably an alkylene chain that is linear or branched or that possibly comprises rings and/or unsaturations, containing from 1 to 40 carbon atoms, in particular from 1 to 20 carbon atoms and better still from 2 to 6 carbon atoms, in particular 6 carbon atoms.
galactomannans containing from one to six and in particular from two to four hydroxyl groups per saccharide, substituted with a saturated or unsaturated alkyl chain, for instance guar gum alkylated with C₁-C₆ and in particular C₁-C₃ alkyl chains, and mixtures thereof;
hydrocarbon-based block copolymers, which are preferably amorphous, formed by polymerization of an olefin. The olefin may especially be an elastomeric ethylenically unsaturated monomer.

Examples of olefins that may be mentioned include ethylenic carbide monomers especially containing one or two ethylenic unsaturations and containing from 2 to 5 carbon atoms, such as ethylene, propylene, butadiene or isoprene.

The polymeric oil-thickening agent is capable of thickening or gelling the organic phase of the composition. The term “amorphous” means a polymer that does not have a crystalline form. The polymeric thickener may also be film-forming, i.e. it is capable of forming a film when applied to the skin.

The polymeric oil-thickening agent may especially be a diblock, triblock, multiblock, radial or star copolymer, or mixtures thereof.

Such polymeric thickeners are described in patent application US-A-2002/005 562 and in patent U.S. Pat. No. 5,221,534.

Advantageously, the polymeric oil-thickening agent is an amorphous block copolymer of styrene and of olefin.

The polymeric oil-thickening agent is preferably hydrogenated to reduce the residual ethylenic unsaturations after polymerization of the monomers.

In particular, the polymeric oil-thickening agent is a copolymer, which is optionally hydrogenated, containing styrene blocks and ethylene/C₃-C₄ alkylene blocks. Diblock copolymers, which are preferably hydrogenated, that may be mentioned include styrene-ethylene/propylene copolymers, styrene-ethylene/butadiene copolymers and styrene-ethylene/butylene copolymers. Diblock copolymers are especially sold under the name Krato® G1701E by the company Kraton Polymers.

Triblock copolymers, which are preferably hydrogenated, that may be mentioned include styrene-ethylene/propylene-styrene copolymers, styrene-ethylene/butadiene-styrene copolymers, styrene-isoprene-styrene copolymers and styrene-butadiene-styrene copolymers. Triblock polymers are especially sold under the names Kraton® G1650, Kraton® G1652, Kraton® D1101, Kratore D1102 and Kraton® D1160 by the company Kraton Polymers.

A styrene-ethylene/butylene-styrene triblock copolymer may especially be used.

According to one preferred embodiment of the invention, a mixture of a styrene-butylene/ethylene-styrene triblock copolymer and of a styrene-ethylene/butylene diblock copolymer, sold under the name Kraton® G1657M by the company Kraton Polymers, may especially be used.
It is also possible to use a mixture of hydrogenated styrene-butylene/ethylene-styrene triblock copolymer and of hydrogenated ethylene-propylene-styrene star polymer, such a mixture being especially in isododecane. Such mixtures are sold, for example, by the company Penreco under the trade names Versagel® M5960 and Versagel® M5670.

Among the lipophilic gelling agents that may be used in the compositions according to the invention, mention may also be made of fatty acid esters of dextrin, such as dextrin palmitates, especially the products sold under the name Rheopearl TL® or Rheopearl KL® by the company Chiba Flour.

Preferably, the composition advantageously comprises a lipophilic gelling polymer chosen from silicone polyamides of the polyorganosiloxane type, hydrocarbon-based block copolymers formed by polymerization of an olefin, and polycondensates of polyamide type, as described above, and mixtures thereof.

This lipophilic gelling polymer is advantageously present in a content ranging from 0.1% to 10% by weight, preferably from 0.2% to 5% by weight and better still from 0.5% to 3% by weight relative to the total weight of the composition.

Even more preferably, these lipophilic gelling polymers are present in the composition according to the invention in a resin/lipophilic polymer ratio ranging from 50/50 to 99/1, preferably from 60/40 to 75/25 and better still from 65/35 to 75/25.

According to one preferred embodiment, a subject of the invention is a composition comprising an aqueous phase, at least one resin chosen from rosins, rosin derivatives and hydrocarbon-based resins, and mixtures thereof, the said resin having a number-average molecular weight of less than or equal to 10 000, and at 1 east one copolymer, which is optionally hydrogenated, containing styrene blocks and ethylene/C₃-C₄ alkylene blocks, the said composition being capable of forming a film with a water resistance such that ΔL is less than or equal to −1.

The block copolymer is advantageously chosen from styrene-ethylene/propylene, styrene-ethylene/butadiene or styrene-ethylene/butylene diblock copolymers, which are optionally hydrogenated, and styrene-ethylene/butadiene-styrene, styrene-butylene/ethylene-styrene, styrene-isoprene-styrene and styrene-butadiene-styrene triblock copolymers, which are optionally hydrogenated. The lipophilic gelling polymer is advantageously a mixture of hydrogenated styrene-butylene/ethylene-styrene triblock copolymer and of styrene-ethylene/butylene diblock copolymer.

The block copolymer is preferably present in the composition according to the invention in a resin/block copolymer ratio ranging from 50/50 to 99/1, preferably from 60/40 to 75125 and better still from 65/35 to 75/25.

Film-Forming Polymer

According to one embodiment, the composition according to the invention may comprise at least one film-forming polymer.

The film-forming polymer may be present in the composition according to the invention in a solids (or active material) content ranging from 0.1% to 30% by weight, preferably from 0.5% to 20% by weight and better still from 1% to 15% by weight relative to the total weight of the composition.

In the present invention, the expression “film-forming polymer” means a polymer that is capable, by itself or in the presence of an auxiliary film-forming agent, of forming a macroscopically continuous film that adheres to the keratin fibres, preferably a cohesive film and better still a film whose cohesion and mechanical properties are such that the said film can be isolated and manipulated separately, for example when the said film is made by casting on a non-stick surface, for instance a Teflon-coated or silicone-coated surface.

Among the film-forming polymers that may be used in the composition of the present invention, mention may be made of synthetic polymers, of free-radical type or of polycondensate type, and polymers of natural origin, and mixtures thereof.

The expression “free-radical film-forming polymer” means a polymer obtained by polymerization of unsaturated and especially ethylenically unsaturated monomers, each monomer being, capable of homopolymerizing (unlike polycondensates).

The film-forming polymers of free-radical type may be, in particular, vinyl polymers or copolymers, in particular acrylic polymers.

The vinyl film-forming polymers may result from the polymerization of ethylenically unsaturated monomers containing at least one acidic group and/or esters of these acidic monomers and/or amides of these acidic monomers.

Monomers bearing an acidic group which may be used are α,β-ethylenic unsaturated carboxylic acids such as acrylic acid, methacrylic acid, crotonic acid, maleic acid or itaconic acid. (Meth)acrylic acid and crotonic acid are preferably used, and more preferably (meth)acrylic acid.

The esters of acidic monomers are advantageously chosen from (meth)acrylic acid esters (also known as (meth)acrylates), especially (meth)acrylates of an alkyl, in particular of a C₁-C₃₀ and preferably C₁-C₂₀ alkyl, (meth)acrylates of an aryl, in particular of a C₆-C₁₀ aryl, and (meth)acrylates of a hydroxyalkyl, in particular of a C₂-C₆ hydroxyalkyl.

Among the alkyl (meth)acrylates that may be mentioned are methyl methacrylate, ethyl methacrylate, butyl methacylate, isobutyl methacrylate, 2-ethylhexyl methacylate, lauryl methacrylate and cyclohexyl methacrylate.

Among the hydroxyalkyl (meth)acrylates that may be mentioned are hydroxyethyl acrylate, 2-hydroxypropyl acrylate, hydroxyethyl methacrylate and 2-hydroxypropylmethacrylate.

Among the aryl (meth)acrylates, that may be mentioned are benzyl acrylate and phenyl acrylate.

The (meth)acrylic acid esters that are particularly preferred are the alkyl (meth)acrylates.

According to the present invention, the alkyl group of the esters may be either fluorinated or perfluorinated, i.e. some or all of the hydrogen atoms of the alkyl group are substituted with fluorine atoms.

Examples of amides of the acid monomers that may be mentioned are (meth)acrylamides, and especially N-alkyl(meth)acrylamides, in particular of a C₂-C₁₂ alkyl. Among the N-alkyl(meth)acrylamides that may be mentioned are N-ethylacrylamide, N-t-butylacrylamide, N-t-octylacrylamide and N-undecylacrylamide.

The vinyl film-forming polymers may also result from the hoxnopolymerization or copolymerization of monomers chosen from vinyl esters and styrene monomers. In particular, these monomers may be polymerized with acid monomers and/or esters thereof and/or amides thereof, such am those mentioned above.

Examples of vinyl esters that may be mentioned are vinyl acetate, vinyl neodecanoate, vinyl pivalate, vinyl benzoate and vinyl t-butylbenzoate.

Styrene monomers that may be mentioned are styrene and α-methylstyrene.

Among the film-forming polycondensates that may be mentioned are polyurethanes, polyesters, polyesteramides, polyamides, epoxyester resins and polyureas.

The polyurethanes may be chosen from anionic, cationic, nonionic and amphoteric polyurethanes, polyurethane-acrylics, polyurethane-polyvinylpyrrolidones, polyester-polyurethanes, polyether-polyurethanes, polyureas and polyurea/polyurethanes, and mixtures thereof.

The polyesters may be obtained, in a known manner, by polycondensation of dicarboxylic acids with polyols, in particular diols.

The dicarboxylic acid may be aliphatic, alicyclic or aromatic. Examples of such acids that may be mentioned are: oxalic acid, malonic acid, dimethylmalonic acid, succinic acid, glutaric acid, adipic acid, pimelic acid, 2,2-dimethylglutaric acid, azeleic acid, suberic-acid, sebacic acid, fumaric acid, maleic acid, itaconic acid, phthalic acid, dodecanedioic acid, 1,3-cyclohexanedicarboxylic acid, 1,4-cyclohexanedicaxboxylic acid, isophthalic acid, terephthalic acid, 2,5-norbornanedicarboxylic acid, diglycolic acid, thiodipropionic acid, 2,5-naphthalenedicarboxylic acid or 2,6-naphthalenedicarboxylic acid. These dicarboxylic acid monomers may be used alone or as a combination of at least two dicarboxylic acid monomers. Among these monomers, the ones preferentially chosen are phthalic acid, isophthalic acid and terephthalic acid.

The diol may be chosen from aliphatic, alicyclic and aromatic diols. The diol used is preferably chosen from: ethylene glycol, diethylene glycol, triethylene glycol, 1,3-propanediol, cyclohexanedimethanol and 4-butanediol. Other polyols that may be used are glycerol, pentaerythritol, sorbitol and trimethylolpropane.

The polyesteramides may be obtained in a manner analogous to that of the polyesters, by polycondensation of diacids with diamines or amino alcohols. Diamines that may be used are ethylenediamine, hexamethylenediamine and meta- or para-phenylenediamine. An amino alcohol that may be used is monoethanolamine.

The polyester may also comprise at least one monomer bearing at least one group —SO₃M, with M representing a hydrogen atom, an ammonium ion NH₄⁺ or a metal ion such as, for example, an Na⁺, Li⁺, K⁺, Mg²⁺, Ca²⁺, Cu²⁺, Fe²⁺ or Fe³⁺ ion. A difunctional aromatic monomer comprising such a group —SO₃M may be used in particular.

The aromatic nucleus of the difunctional aromatic monomer also bearing a group —SO₃M as described above may be chosen, for example, from benzene, naphthalene, anthracene, biphenyl, oxybiphenyl, sulfbnylbiphenyl and methylenebiphenyl nuclei. As examples of difunctional aromatic monomers also bearing a group —SO₃M, mention may be made of: sulfoisophthalic acid, sulfoterephthalic acid, sulfophthalic acid, 4-sulfonaphthalene-2,7-dicarboxylic acid.

The copolymers preferably used are those based on isophthalate/sulfoisophthalate, and more particularly copolymers obtained by condensation of diethylene glycol, cyclohexanedimethanol, isophthalic acid and sulfoisophthalic acid.

The polymers of natural origin, optionally modified, may be chosen from shellac resin, sandarac gum, dammar resins, elemi gums, copal resins and cellulose polymers, and mixtures thereof.

According to a first embodiment of the composition according to the invention, the film-forming polymer may be a water-soluble polymer and may be present in an aqueous phase of the composition; the polymer is thus solubillized in the aqueous phase of the composition. Examples of water-soluble film-forming polymers that may be mentioned are:

proteins, for instance proteins of plant origin such as wheat proteins and soybean proteins; proteins of animal origin such as keratins, for example keratin hydrolysates and sulfonic keratins;

polymers of cellulose such as hydroxyethylcellulose, hydroxypropylcellulose, methylcellulose, ethylhydroxyethylcellulose and carboxymethylcellulose, and quaternized cellulose derivatives;

acrylic polymers or copolymers, such as polyacrylates or polymethacrylates;

vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl, acetate; copolymers of vinylpyrrolidone and of caprolactam; polyvinyl alcohol;

polymers of natural origin, which are optionally modified, such as:

gum arabics, guar gum, xanthan derivatives, karaya gum;

alginates and carrageenans;

glycosaminoglycans, hyaluronic acid and derivatives thereof;

shellac resin, sandarac gum, dammar resins, elemi gums and copal resins;

deoxyribonucleic acid;

mucopolysaccharides such as chondroitin sulfate,

and mixtures thereof.

According to another embodiment of the composition according to the invention, the film-forming polymer may be a polymer dissolved in a liquid fatty phase comprising organic solvents or oils such as those described above (the film-forming polymer is thus said to be a liposoluble polymer). For the purposes of the invention, the expression “liquid fatty phase” means a fatty phase which is liquid at room temperature (25° C.) and atmospheric pressure (760 mmHg, i.e. 10⁵ Pa), composed of one or more fatty substances that are liquid at room temperature, such as the oils described above, which are generally mutually compatible.

The liquid fatty phase preferably comprises a volatile oil, optionally mixed with a non-volatile oil, the oils possibly being chosen from those mentioned above.

Examples of liposoluble polymers which may be mentioned are copolymers of vinyl ester (the vinyl group being directly linked to the oxygen atom of the ester group and the vinyl ester containing a saturated, linear or branched hydrocarbon-based radical of 1 to 19 carbon atoms, linked to the carbonyl of the ester group) and of at least one other monomer which may be a vinyl ester (other than the vinyl ester already present), an α-olefin (containing from 8 to 28 carbon atoms), an alkyl vinyl ether (in which the alkyl group comprises from 2 to 18 carbon atoms) or an allylic or methallylic ester (containing a saturated, linear or branched hydrocarbon-based radical of 1 to 19 carbon atoms, linked to the carbonyl of the ester group).

These copolymers may be crosslinked with the aid of crosslinking agents, which may be either of the vinyl type or of the allylic or methallylic type, such as tetraallyloxyethane, divinylbenzene, divinyl octane-dioate, divinyl dodecanedioate and divinyl octadecane-dioate.

Examples of these copolymers that may be mentioned are the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/acetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

Examples of liposoluble film-forming polymers which may also be mentioned are liposoluble copolymers, and in particular those resulting from the copolymerization of vinyl esters containing from 9 to 22 carbon atoms or of alkyl acrylates or methacrylates, and alkyl radicals containing from 10 to 20 carbon atoms.

Such liposoluble copolymers may be chosen from polyvinyl stearate, polyvinyl stearate crosslinked with the aid of divinylbenzene, of diallyl ether or of diallyl phthalate, polystearyl (meth)acrylate, polyvinyl laurate and polylauryl (meth)acrylate, it being possible for these poly(meth)acrylates to be cross-linked with the aid of ethylene glycol dimethacrylate or tetraethylene glycol dimethacrylate.

The liposoluble copolymers defined above are known and are described in particular in patent application FR-A-2 232 303; they may have a weight-average molecular weight ranging from 2000 to 500 000 and preferably from 4000 to 200 000.

As liposoluble film-forming polymers which may be used in the invention, mention may also be made of polyalkylenes and in particular copolymers of C₂-C₂₀ alkenes, such as polybutene, alkylcelluloses with a linear or branched, saturated or unsaturated C₁-C₈ alkyl radical, for instance ethylcellulose and propylcellulose, copolymers of vinylpyrrolidone (VP) and in particular copolymers of vinylpyrrolidone and of C₂ to C₄₀ and better still C₃ to C₂₀ alkene. As examples of VP copolymers which may be used in the invention, mention may be made of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

Mention may also be made of silicone resins, which are generally soluble or swellable in silicone oils, which are crosslinked polyorganosiloxane polymers. The nomenclature of silicone resins is known under the name “MDTQ”, the resin being described as a function of the various siloxane monomer units it comprises, each of the letters “MDTQ” characterizing, a type of unit.

Examples of commercially available polymethylsilsesquioxane resins that may be mentioned include those sold:

by the company Wacker under the reference Resin MK, such as Belsil PMS MK;

by the company Shin-Etsu under the reference KR-220L.

Siloxysilicate resins that may be mentioned include trimethyl siloxysilicate (TMS) resins such as those sold under the reference SR 1000 by the company General Electric or under the reference TMS 803 by the company Wacker. Mention may also be made of the trimethyl siloxysilicate resins sold in a solvent such as cyclomxethicone, sold under the name KF-7312J by the company Shin-Etsu, and DC 749 and DC 593 by the company Dow Corning.

Mention may also be made of silicone resin copolymers such as those mentioned above with polydimethylsiloxaxes, for instance the pressure-sensitive adhesive copolymers sold by the company Dow Corning under the reference Bio-PSA and described in document U.S. Pat. No. 5,162,410, or the silicone copolymers derived from the reaction of a silicone resin, such as those described above, and of a diorganosiloxane, as described in document WO 2004/073 626.

According to one embodiment of the invention, the film-forming polymer is a film-forming linear block ethylenic polymer, which preferably comprises at least a first block and at least a second block with different glass transition temperatures (Tg), the said first and second blocks being linked together via an intermediate block comprising at least one constituent monomer of the first block and at least one constituent monomer of the second block.

Advantageously, the first and second blocks of the block polymer are mutually incompatible.

Such polymers are described, for example, in document EP 1 411 069 or WO 04/028 488.

The film-forming polymer may also be present in the composition in the form of particles dispersed in an aqueous phase or in a non-aqueous solvent phase, which is generally known as a latex or pseudolatex. The techniques for preparing these dispersions are well known to those skilled in the art.

Aqueous dispersions of film-forming polymers that may be used include the acrylic dispersions sold under the names Neocryl XK-90®, Neocryl A-1070®, Neocryl A-1090®, Neocryl BT-62®, Neocryl A-1079® and Neocryl A-523® by the company Avecia-Neoresins, Dow Latex 432® by the company Dow Chemical, Daitosol 5000 AD® or Daitosol 5000 SJ® by the company Daito Kasey Kogyo; Syntran 5760® by the company Interpolymer, or the aqueous dispersions of polyurethane sold under the names Neorez R-981® and Neorez R-974® by the company Avecia-Neoresins, Avalure UR-405®, Avalure UR-410®, Avalure UR-425®, Avalure UR-450®, Sancure 875®, Sancure 861®, Sancure 878® and Sancure 2060® by the company Goodrich, Impranil 85® by the company Bayer and Aquamere H-1511® by the company Hydromer; the sulfopolyesters sold under the brand name Eastman AQ® by the company Eastman Chemical Products, and vinyl dispersions, for instance Mexomer PAM® from the company Chimex, and mixtures thereof.

Examples of non-aqueous film-forming polymer dispersions that may also be mentioned include acrylic dispersions in isododecane, for instance Mexomer PAP® from the company Chimex, and dispersions of particles of a grafted ethylenic polymer, preferably an acrylic polymer, in a liquid fatty phase, the ethylenic polymer advantageously being dispersed in the absence of additional stabilizer at the surface of the particles as described especially in document WO 04/055 081.

The composition according to the invention may comprise a plasticizer that promotes the formation of a film with the film-forming polymer. Such a plasticizer may be chosen from any compound known to those skilled in the art as being capable of fulfilling the desired function.

Dyestuff

The composition according to the invention may also comprise at least one dyestuff, for instance pulverulent dyes, liposoluble dyes and water-soluble dyes.

The pulverulent dyestuffs may be chosen from pigments and nacres.

The pigments may be white or coloured, mineral and/or organic, and coated or uncoated. Among the mineral pigments that may be mentioned are titanium dioxide, optionally surface-treated, zirconium oxide, zinc oxide or cerium oxide, and also iron oxide or chromium oxide, manganese violet, ultramarine blue, chromium hydrate and ferric blue. Among the organic pigments that may be mentioned are carbon black, pigments of D&C type, and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica with, especially, ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C orange 5, quinoline yellow and annatto.

These dyestuffs may be present in a content ranging from 0.01% to 30% by weight relative to the total weight of the composition.

Fillers

The composition according to the invention may also comprise at least one filler.

The fillers may be chosen from those that are well known to those skilled in the art and commonly used in cosmetic compositions. The fillers may be mineral or organic, and lamellar or spherical. Mention may be made of talc, mica, silica, kaolin, polyamide powders, for instance the Nylon® sold under the trade name Orgasol® by the company Atochem, poly-β-alanine powders and polyethylene powders, powders of tetrafluoroethylene polymers, for instance Teflon®, lauroyllysine, starch, boron nitride, expanded polymeric hollow microspheres such as those of polyvinylidene chloride/acrylonitrile, for instance the products sold under the name Expancel® by the company Nobel Industrie, acrylic powders, such as those sold under the name Polytrap® by the company Dow Corning, polymethyl methacrylate particles and silicone resin microbeads (for example Tospearls® from Toshiba), precipitated calcium carbonate, magnesium carbonate and magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms and in particular from 12 to 18 carbon atoms, for example zinc, magnesium or lithium stearate, zinc laurate and magnesium myristate.

It is also possible to use a compound that is capable of swelling on heating, and especially heat-expandable particles such as non-expanded microspheres of copolymer of vinylidene chloride/acrylonitrile/methyl methacrylate or of acrylonitrile homopolymer copolymer, for instance those sold, respectively, under the references Expancel® 820 DU 40 and Expancel® 007WU by the company Akzo Nobel.

The fillers may represent from 0.1% to 25% and in particular from 1% to 20% by weight relative to the total weight of the composition.

The composition of the invention may also comprise any additive usually used in cosmetics, such as antioxidants, preserving agents, fibres, fragrances, neutralizers, gelling agents, thickeners, vitamins, coalescers and plasticizers, and mixtures thereof.

Fibres

The composition according to the invention may also comprise fibres to allow an improvement in the lengthening effect.

The term “fibre” should be understood as meaning an object of length L and diameter D such that L is very much greater than D, D being the diameter of the circle in which the cross section of the fibre is inscribed. In particular, the ratio L/D (or shape factor) is chosen in the range from 3.5 to 2500, especially from 5 to 500 and in particular from 5 to 150.

The fibres that may be used in the composition of the invention may be mineral or organic fibres of synthetic or natural origin. They may be short or long, individual or organized, for example braided, and hollow or solid. They may have any shape, and may especially have a circular or polygonal (square, hexagonal or octagonal) cross section, depending on the intended specific application. In particular, their ends are blunt and/or polished to prevent injury.

In particular, the fibres have a length ranging from 1 μm to 10 mm, preferably from 0.1 mm to 5 mm and better still from 0.3 mm to 3.5 mm. Their cross section may be within a circle of diameter ranging from 2 nm to 500 μm, preferably ranging from 100 nm to 100 μm and better still from 1 μm to 50 μm. The weight or yarn count of the fibres is often given in denier or decitex, and represents the weight in grams per 9 km of yarn. In particular, the fibres according to the invention may have a yarn count chosen in the range from 0.15 to 0.30 denier and better still from 0.18 to 18 denier.

The fibres that may be used in the composition of the invention may be chosen from rigid or non-rigid fibres, and may be of synthetic or natural, mineral or organic origin.

Moreover, the fibres may or may not be surface-treated, may be coated or uncoated, and may be coloured or uncoloured.

As fibres that may be used in the composition according to the invention, mention may be made of non-rigid fibres such as polyamide (Nylon®) fibres or rigid fibres such as polyimideamide fibres, for instance those sold under the names Kermel® and Kermel Tech® by the company Rhodia or poly(p-phenyleneterephthalamide) (or aramid) fibres sold especially under the name Kevlar® by the company DuPont de Nemours.

The fibres may be present in the composition according to the invention in a content ranging from 0.01% to 10% by weight, in particular from 0.1% to 5% by weight and more particularly from 0.3% to 3% by weight relative to the total weight of the composition.

Cosmetic Active Agents

As cosmetic active agents that may be used in the compositions according to the invention, mention may be made especially of antioxidants, preserving agents, fragrances, neutralizers, emollients, moisturizers, vitamins and screening agents, in particular sunscreens.

Needless to say, a person skilled in the art will take care to select the optional additional additives and/or the amount thereof such that the advantageous properties of the composition according to the invention are not, or are not substantially, adversely affected by the envisaged addition.

The compositions according to the invention may be prepared according to methods known to those skilled in the art.

The composition is preferably prepared in the following manner:

the fatty phase is prepared by mixing the waxes and optionally the oils while heating at 95° C., with the ground pigments and the surfactants,

the aqueous phase is prepared by mixing the water, optionally the surfactants and the film-forming polymers,

the aqueous phase is added to the fatty phase at 95° C. with mechanical stirring over about 20 minutes, and the mixture is then cooled to about 40° with continued stirring,

a gel, prepared beforehand by mixing the resin, optionally the gelling agent for the oily phase and some of the organic solvents while heating the whole at a temperature not exceeding the evaporation point of the organic solvent (or of the solvent mixture), is added to this mixture, at 40° C.

The composition according to the invention may be packaged in a container delimiting at least one compartment that comprises the said composition, the said container being closed by a closing member.

The container is preferably associated with an applicator, especially in the form of a brush comprising an arrangement of bristles maintained by a twisted wire. Such a twisted brush is especially described in patent. U.S. Pat. No. 4,887,622. It may also be in the form of a comb comprising a plurality of application members, obtained especially by moulding. Such combs are described, for example, in patent FR 2 796 529. The applicator may be solidly attached to the container, as described, for example, in patent FR 2 761 959. Advantageously, the applicator is solidly attached to a stem, which is itself solidly attached to the closing member.

The closing member may be coupled to the container by screwing. Alternatively, the coupling between the closing member and the container takes place other than by screwing, especially via a bayonet mechanism, by click-fastening or by tightening. The term “click-fastening” in particular means any system involving the passing of a rim or bead of material by elastic deformation of a portion, especially of the closing member, followed by return to the elastically unstressed position of the said portion after the rim or bead has been passed.

The container may be at least partly made of thermoplastic material. Examples of thermoplastic materials that may be mentioned include polypropylene and polyethylene.

Alternatively, the container is made of a non-thermoplastic material, especially of glass or metal (or alloy).

The container is preferably equipped with a drainer located in the region of the aperture of the container. Such a drainer makes it possible to wipe the applicator and, optionally, the stem to which it may be solidly attached. Such a drainer is described, for example, in patent FR 2 792 618.

The content of the patents or patent applications mentioned previously are incorporated by reference into the present patent application.

The examples that follow are presented as non-limiting illustrations of the invention. Unless otherwise indicated, the amounts are given in grams.

### EXAMPLE 1

The mascara below according to the invention is prepared:

| | |
|---|---|
| Carnauba wax | 5.75 |
| Rice bran wax | 5.87 |
| Candelilla wax | 1.97 |
| Esters of hydrogenated olive oil and of | 4.96 |
| stearyl alcohol (Phytowax Olive 18 L 57 | |
| from Sophim) | |
| Gum arabic | 1.52 |
| Hydroxyethyl cellulose | 0.22 |
| Styrene/methylstyrene/indene hydrogenated | 2 |
| copolymer (Regalite R1100 from Eastman) | |
| Polyvinyl alcohol | 0.2 |
| Oxyethylenated (20 EO) oxypropylenated (20 PO) | 0.2 |
| polydimethylsiloxane (DC 2-5520) from | |
| Dow Corning) | |
| Isododecane | 3 |
| Simethicone | 0.12 |
| Black iron oxide | 8 |
| Stearic acid | 5.45 |
| Triethanolamine | 2.4 |
| Preserving agents | qs |
| Water | qs 100 |

The water resistance of the composition was measured according to the measuring method indicated previously in the description.

The resistance to rubbing of the mascara is evaluated as follows:

The composition is applied to 3 samples of straight 30-knots Caucasian hair (60 eyelashes 1 cm long), 2 cm fringe length, by performing three series of 10 sweeps at 2-minute intervals, with uptake of product between each series of 10.

Each sample is then dried at room temperature for a drying time of one hour.

Each sample is then rubbed 30 times with a hard brush, and the debris is recovered on adhesive paper and evaluated visually according to the following notation:

0=no grains

1=very few grains

2=few grains

3=quite a few grains

4=many grains

5=very many grains

The following results are obtained:

| | |
|---|---|
| Water resistance (ΔL) | −5.84 |
| Resistance to rubbing (grade) | 1-2 (few grains) |

### EXAMPLES 2 AND 3

The mascaras of Examples 2 and 3 below according to the invention are prepared:

| | Example 2 | Example 3 |
|---|---|---|
| | (invention) | (invention) |
| Carnauba wax | 4.2 | 4.2 |
| Rice bran wax | 4.29 | 4.29 |
| Candelilla wax | 1.44 | 1.44 |
| Esters of hydrogenated olive | 3.62 | 3.62 |
| oil and of stearyl alcohol | | |
| (Phytowax Olive 18 L 57 from | | |
| Sophim) | | |
| Gum arabic | 1.52 | 1.52 |
| Hydroxyethylcellulose | 0.22 | 0.22 |
| Ethylene-styrene/butylene- | 1 | 1 |
| styrene copolymer (Kraton | | |
| G1657M from Shell) | | |
| Styrene/methylstyrene/indene/ | 2 | 3 |
| styrene hydrogenated copolymer | | |
| Regalite R1100 from Eastman) | | |
| Polyvinyl alcohol | 0.2 | 0.2 |
| Oxyethylenated (20 EO) | 0.2 | 0.2 |
| oxypropylenated (20 PO) | | |
| polydimethylsiloxane (DC 2- | | |
| 5520) from Dow Corning) | | |
| Isododecane | 7 | 6 |
| Simethicone | 0.12 | 0.12 |
| Black iron oxide | 8 | 8 |
| Stearic acid | 5.45 | 5.45 |
| Triethanolamine | 2.4 | 2.4 |
| Preserving agents | qs | qs |
| Water | qs 100 | qs 100 |

### Procedure:

A pregel is prepared by mixing the resin (Regalite), some of the isododecane and the lipophilic gelling polymer (Kraton) in the isododecane while heating at 30-35° C. with stirring.

In parallel, the waxes are heated to about 95° C., followed by addition of the preground pigments and the stearic acid, with stirring of the whole using a Rayneri blender for about 20 minutes. The water, the polyvinyl alcohol, the hydroxyethylcellulose and the gum arabic are then added; the mixture is cooled to 40° C. with mechanical stirring, and the rest of the isododecane and the pregel are then added.

For each composition, the water resistance was measured according to the measuring method indicated previously in the description.

The resistance to rubbing is evaluated according to the protocol described in Example 1 for compositions 2 and 3 and also for the “control” composition (outside the invention) described at the start of the present description.

The following results are obtained:

| | Example 2 | Example 3 |
|---|---|---|
| Water resistance (ΔL) | −4.76 | −4.46 |

| | | | Control |
|---|---|---|---|
| | Example 2 | Example 3 | composition |
| Resistance to rubbing | 1 (no | 1 (very | 4 (very many |
| | grains) | few grains) | grains) |

It is found that the mascaras of Examples 2 and 3 according to the invention have good water resistance and also better resistance to rubbing than the mascara not comprising resin (control composition).

1. Composition for coating keratin fibres, comprising an aqueous phase and at least one resin chosen from rosins, rosin derivatives and hydrocarbon-based resins, and mixtures thereof, the said resin having a number-average molecular weight of less than or equal to 10 000, the said composition being capable of forming a film having a water resistance such that ΔL is less than or equal to −1.

2. Composition according to claim 1, characterized in that it has a water resistance such that ΔL ranges from −1 to −7, and ΔL is preferably less than or equal to −2.

3. Composition according to claim 1 or 2, characterized in that the resin has a number-average molecular weight of less than or equal to 5000, preferably less than or equal to 2000 and better still less than or equal to 1000.

4. Composition according to one of claims 1 to 3, characterized in that the hydrocarbon-based resin is chosen from indene resins, aliphatic pentanediene resins, mixed resins of pentanediene and of indene, diene resins of cyclopentadiene dimers and diene resins of isoprene dimers, and mixtures thereof.

5. Composition according to one of the preceding claims, characterized in that the hydrocarbon-based resin is chosen from:
indene hydrocarbon-based resins derived from the polymerization in major proportion of indene monomer and in minor proportion of monomers chosen from styrene, methylindene and methylstyrene, and mixtures thereof;
aliphatic pentanediene resins derived from the majority polymerization of the trans or cis-piperylene (1,3-pentanediene) monomer and of minor monomers chosen from isoprene, butene, 2-methyl-2-butene, pentene and 1,4-pentanediene, and mixtures thereof;
mixed resins of pentanediene and of indene, which are derived from the polymerization of a mixture of pentanediene and indene monomers;
diene resins of cyclopentadiene dimers such as those derived from the polymerization of first monomers chosen from indene and styrene, and of second monomers chosen from dicyclopentadiene, methyldicyclopentadiene and other pentanediene dimers, and mixtures thereof;
diene resins of isoprene dimers such as terpenic resins derived from the polymerization of at least one monomer chosen from α-pinene, β-pinene and limonene, and mixtures thereof;
and mixtures thereof.

6. Composition according to one of the preceding claims, characterized in that the resin is chosen from indene hydrocarbon-based resins derived from the polymerization in major proportion of indene monomer and in minor proportion of monomers chosen from styrene, methylindene and methylstyrene, and mixtures thereof.

7. Composition according to claim 6, characterized in that the indene hydrocarbon-based resin is hydrogenated.

8. Composition according to one of the preceding claims, characterized in that the resin is chosen from indene/methylstyrene/styrene hydrogenated copolymers.

9. Composition according to any one of the preceding claims, characterized in that the resin is present in a content ranging from 0.1% to 20% by weight, preferably from 0.5% to 15% by weight and better still from 1% to 10% by weight relative to the total weight of the composition.

10. Composition according to any one of the preceding claims, characterized in that the aqueous phase comprises water and/or at least one water-soluble solvent.

11. Composition according to claim 10, characterized in that the aqueous phase is present in a content ranging from 5% to 95% by weight, preferably ranging from 10% to 80% by weight and preferentially ranging from 15% to 60% by weight relative to the total weight of the composition.

12. Composition according to any one of the preceding claims, characterized in that it comprises an emulsifying system.

13. Composition according to one of the preceding claims, characterized in that it comprises a liquid fatty phase.

14. Composition according to one of the preceding claims, characterized in that the liquid fatty phase comprises at least one hydrocarbon-based volatile oil containing from 8 to 16 carbon atoms.

15. Composition according to claim 14, characterized in that the liquid fatty phase represents from 0.5% to 30% by weight, preferably from 1% to 20% and even more preferably from 2% to 10% by weight relative to the total weight of the composition.

16. Composition according to any one of the preceding claims, characterized in that it comprises at least one structuring agent for the oily phase or organic solvent, chosen from waxes, semi-crystalline polymers and lipophilic gelling agents, and mixtures thereof.

17. Composition according to claim 16, characterized in that the structuring agent represents from 0.05% to 70% by weight, preferably from 0.1% to 40% by weight, better still from 1% to 30% by weight and even more preferably from 1.5% to 15% by weight relative to the total weight of the composition.

18. Composition according to any one of the preceding claims, characterized in that it comprises a lipophilic gelling polymer chosen from:
polycondensates of polyamide type resulting from condensation between (α) at least one acid chosen from dicarboxylic acids containing at least 32 carbon atoms and (β) an alkylenediamine, in which the polyamide polymer comprises at least one carboxylic acid end group esterified or amidated with at least one linear, saturated monoalcohol or monoamine containing from 12 to 30 carbon atoms;
silicone polymers of the type such as:
1) polyorganosiloxanes comprising at least two groups capable of establishing hydrogen interactions, these two groups being located in the polymer chain, and/or
2) polyorganosiloxanes comprising at least two groups capable of establishing hydrogen interactions, these two groups being located on grafts or branches,
the groups capable of establishing hydrogen interactions possibly being chosen from ester, amide, sulfonamide, carbamate, thiocarbamate, urea, urethane, thiourea, oxamido, guanidino and biguanidino groups, and combinations thereof,
hydrocarbon-based block copolymers formed by polymerization of ethylenic carbide monomers, especially containing one or two ethylenic unsaturations, and containing from 2 to 5 carbon atoms,
and mixtures thereof.

19. Composition according to any one of the preceding claims, characterized in that it comprises a lipophilic gelling polymer chosen from polymers formed by polymerization of styrene and of an olefin chosen from ethylene, propylene, butadiene and isoprene.

20. Composition according to any one of the preceding claims, characterized in that it comprises a lipophilic gelling polymer chosen from copolymers, which are optionally hydrogenated, containing styrene blocks and ethylene/C₃-C₄ alkylene blocks.

21. Composition according to any one of the preceding claims, characterized in that it comprises a lipophilic gelling polymer chosen from styrene-ethylene/propylene, styrene-ethylene/butadiene and styrene-ethylene/butylene diblock copolymers, which are optionally hydrogenated, and styrene-ethylene/butadiene-styrene, styrene-butylene/ethylene-styrene, styrene-isoprene-styrene and styrene-butadiene-styrene triblock copolymers, which are optionally hydrogenated.

22. Composition according to any one of the preceding claims, characterized in that it comprises a lipophilic gelling polymer chosen from mixtures of hydrogenated styrene-butylene/ethylene-styrene triblock copolymer and of styrene-ethylene/butylene diblock copolymer.

23. Composition according to one of claims 16 to 22, characterized in that the lipophilic gelling polymer is present in a resin/lipophilic polymer ratio ranging from 50/50 to 99/1, preferably from 60/40 to 75/25 and better still from 65/35 to 75/25.

24. Composition according to any one of the preceding claims, characterized in that it comprises at least one film-forming polymer.

25. Composition according to claim 24, characterized in that the film-forming polymer is present in a solids content ranging from 0.1% to 30% by weight, preferably from 0.5% to 20% by weight and better still from 1% to 15% by weight relative to the total weight of the composition.

26. Composition according to one of the preceding claims, characterized in that it comprises a dyestuff.

27. Composition according to claim 26, characterized in that the dyestuff represents from 0.01% to 30% by weight relative to the total weight of the composition.

28. Process for making up keratin fibres, characterized in that a composition as defined according to any one of claims 1 to 27 is applied to the said keratin fibres and especially to the eyelashes.

29. Use of at least one resin chosen from rosins, rosin derivatives and hydrocarbon-based resins, and mixtures thereof, in a keratin fibre coating composition comprising a continuous aqueous phase, to obtain a composition capable of forming a file which, when deposited on keratin fibres, has a water resistance such that ΔL is less than or equal to −1.
